(19) United States
(12) Patent Application Publication
(10) Pub. No.: US 2025/0332177 A1
(43) Pub. Date: Oct. 30, 2025

(54) Methylene Blue COVID-19 Composition

(71) Applicant: Dorra Frikha, Wayne, NJ (US)

(72) Inventor: Dorra Frikha, Wayne, NJ (US)

(21) Appl. No.: 18/647,117

(22) Filed: Apr. 26, 2024

(51) Int. Cl. A61K 31/616; A61K 9/14; A61K 31/5415

(52) U.S. Cl. CPC A61K 31/616; A61K 9/14; A61K 31/5415

(57) ABSTRACT

A methylene blue COVID-19 composition for treating short-term and long-term COVID-19 symptoms. The methylene blue COVID-19 composition consists of methylene blue and aspirin. The amount of methylene blue in the overall composition is in the range of about 50 mg to 300 mg, while the amount of aspirin in the overall composition is in the range of about 80 mg to 350 mg. The methylene blue COVID-19 composition is formed as a powder that is dissolvable in water or another beverage for patient consumption. The overall composition is used as a treatment for up to about 6 months after testing positive for a COVID-19 infection, in order to effectively treat various symptoms of COVID-19 infections. The methylene blue COVID-19 composition is dissolved in liquid to be a standalone methylene blue COVID-19 solution. In one embodiment, the methylene blue COVID-19 composition is in a pill form.

### BACKGROUND OF THE INVENTION

The present invention relates to a COVID-19 composition. More specifically, the present invention relates to a methylene blue COVID-19 composition.

COVID-19 or Coronavirus Disease 2019 and other airborne diseases may be incredibly dangerous to those who get infected with the illness. Even if the individual who is infected by these airborne diseases may recover from the illness, they may be left with serious residual effects. These residual effects that come following an airborne disease infection may last for a prolonged period. Additionally, some individuals may never fully recover from the airborne disease or its residual effects. Moreover, some individuals will experience additional symptoms like fatigue, anxiety, depression, headaches, cognitive difficulties, stroke, pain, and sleep disorders and the like. Also, some of the individuals that contract the airborne disease once, or even receive a vaccine, may become reinfected with the illness.

The standard available treatment methods may be ineffective in limiting the symptoms that an infected individual experiences. In order to address these concerns, the present invention provides users with a methylene blue COVID-19 composition for treating short-term and long-term COVID-19 symptoms.

### SUMMARY OF THE INVENTION

In view of the foregoing disadvantages inherent in the known types of methylene blue COVID-19 composition now present in the prior art, the present invention provides a methylene blue COVID-19 composition wherein the same may be utilized for providing convenience for a user for treating short-term and long-term COVID-19 symptoms.

The present invention includes a methylene blue COVID-19 composition that consists of a quantity of methylene blue and a quantity of aspirin.

The present invention includes a methylene blue COVID-19 composition that consists of a quantity of methylene blue that is in the range of about 50 mg to 300 mg and a quantity of aspirin that is in the range of about 80 mg to 350 mg.

The present system comprises a methylene blue COVID-19 composition that consists of a methylene blue COVID-19 composition that is preferably formed as a powder that is dissolvable in water or another beverage for patient consumption.

The present invention includes a methylene blue COVID-19 composition that is used as a treatment for up to about 6 months after testing positive for a COVID-19 infection, in order effectively treat various symptoms of COVID-19 infections.

The present system comprises a methylene blue COVID-19 composition that consists of a methylene blue COVID-19 composition that is dissolved in liquid to be a standalone methylene blue COVID-19 solution.

The present system comprises a methylene blue composition for treating COVID-19 that the methylene blue COVID-19 composition is in a pill form.

### DETAILED DESCRIPTION OF THE INVENTION

The methylene blue COVID-19 composition may include a quantity of methylene blue and a quantity of aspirin.

The quantity of methylene blue or Methylthionine chloride within the treatment method composition may be in the range of about 50 mg to 300 mg. The quantity of methylene blue may counteract metabolic oxidative stress responsible of the pathogenesis of COVID-19. It is believed that methylene blue may have the ability to target immune system cells and molecular mechanisms responsible for triggering the adequate body response to viral infection. Moreover, methylene blue may target the central nervous system as a promising and effective strategy for preventing or treating neuropsychiatric symptoms. The methylene blue may be able to maintain normal physiological pathways under stress conditions associated with significant accumulation of oxidative stress including infection or the like.

Metabolic oxidative stress may be developed when there is malfunctioned mitochondria and alteration in cellular energy supply. Methylene blue may have the ability to counteract the mitochondria function and maintain a cell's required Adenosine Triphosphate or ATP levels. Methylene blue may also inhibit a SARS-CoV-2 protein-protein interaction. The SARS-CoV-2 protein-protein interaction may contribute to anti-viral activity that may lead to a change in intracellular Potential of Hydrogen or pH and Oxidation-Reduction or REDOX state.

The quantity of aspirin (2-Acetoxybenzoic acid or acetylsalicylic acid (ASA)) within the treatment method composition may vary in the range between about 80 mg to 350 mg. The quantity of aspirin within that range may reduce the risk of blood clots and help a user's immune system combat viral infection. Moreover, that quantity of aspirin may reduce the levels of interleukin-6 (IL-6). Interleukin-6 (IL-6) may be an immune signaling molecule associated with cytokine storm for COVID-19 patients or the like.

The preferable form of the methylene blue COVID-19 composition may be a powder or the like. The powder may relatively easily dissolve in liquid, specifically water or the like, and provide a relatively easy method of consumption for the user. The consumption of the powder may be used as a treatment for up to about 6 months after testing positive for a COVID-19 infection. When a user with a positive COVID-19 infection consumes the powder, the various symptoms that they might experience because of the infection may be effectively treated and their severity limited.

The methylene blue COVID-19 composition may relatively easily dissolve in liquid to be a standalone methylene blue COVID-19 solution or the like. The liquid may be water or the like.

The combination of methylene blue and aspirin holds the potential for synergistic effects in the context of treating viral infections. Methylene blue, with its broad-spectrum antiviral properties, may target viral replication and inhibit viral entry into host cells. It disrupts viral membrane integrity and inhibits viral protein synthesis. On the other hand, aspirin, with its anti-inflammatory and immunomodulatory properties, can help reduce excessive inflammation associated with viral infections and regulate the immune response. By combining methylene blue and aspirin, the treatment approach may address both the viral replication cycle and the host immune response simultaneously, potentially enhancing the overall antiviral effects. Methylene blue's ability to interfere with viral replication may synergistically complement aspirin's anti-inflammatory action by reducing the viral load and subsequent inflammation. Moreover, aspirin's modulation of the immune response may help optimize the host defense mechanisms against the viral infection.

CH₃COOC₆H₄COOH₄H₂O—C₆H₄(OH)COOH+CH₃COOH (1)

As shown in Equation 2, however, direct reactions between aspirin and methylene blue may not be common unless specific conditions or catalysts are introduced. Both compounds have specific uses and are generally stable in their intended applications.

The reaction between aspirin (acetylsalicylic acid) and methylene blue in aqueous solution depends on various factors such as concentration, temperature, and reaction conditions. However, in a general sense, both compounds are relatively stable under normal conditions. Aspirin is an organic acid, and it can undergo hydrolysis in the presence of water to produce salicylic acid. The reaction involves the cleavage of the ester bond in aspirin.

C₁6H₁₈CLN₃S—C₁6H₁₈N₃S⁺+Cl⁻ (2)

As shown in Equation 2, Methylene blue, being an organic chloride salt, may dissociate in water to form chloride ions and the cationic form of methylene blue.

The foregoing descriptions of specific embodiments of the present invention have been presented for purposes of illustration and description. They are not intended to be exhaustive or to limit the present invention to the precise forms disclosed, and obviously many modifications and variations may be possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A methylene blue COVID-19 composition, consisting of:
a quantity of methylene blue; and
a quantity of aspirin.

2. The methylene blue COVID-19 composition, according to claim 1, wherein the quantity of methylene blue is in the range of about 50 mg to 300 mg.

3. The methylene blue COVID-19 composition, according to claim 1, wherein the methylene blue COVID-19 composition is a powder.

4. The methylene blue COVID-19 composition, according to claim 3, wherein the powder is used as a treatment for up to 6 months after testing positive for a COVID-19 infection.

5. The methylene blue COVID-19 composition, according to claim 1, wherein the quantity of aspirin is in the range of about 80 mg to 350 mg.

6. A methylene blue COVID-19 composition, consisting of:
a quantity of methylene blue in the range of about 50 mg to 300 mg; and
a quantity of aspirin in the range of about 80 mg to 350 mg;
wherein the methylene blue COVID-19 composition is a powder.

7. The methylene blue COVID-19 composition, according to claim 6, wherein the powder dissolves in a liquid to provide an easy method of consumption for a user.

8. The methylene blue COVID-19 composition, according to claim 7, wherein the liquid is water.

9. The methylene blue COVID-19 composition, according to claim 6, wherein the powder is used as a treatment for up to about 6 months after testing positive for a COVID-19 infection.

10. The methylene blue COVID-19 composition, according to claim 6, wherein the quantity of methylene blue counteracts metabolic oxidative stress responsible for a pathogenesis of COVID-19.

11. The methylene blue COVID-19 composition, according to claim 6, wherein the methylene blue targets immune system cells and molecular mechanisms responsible for triggering an adequate body response to viral infection.

12. The methylene blue COVID-19 composition, according to claim 6, wherein the methylene blue maintains normal physiological pathways under stress conditions associated with significant accumulation of oxidative stress including infection.

13. The methylene blue COVID-19 composition, according to claim 6, wherein the quantity of aspirin reduces levels of a plurality of interleukin-6 (IL-6) that is an immune signaling molecule associated with cytokine storm for COVID-19 patients.

14. A methylene blue COVID-19 solution, consisting of:
a quantity of methylene blue in the range of about 50 mg to 300 mg; and
a quantity of aspirin in the range of about 80 mg to 350 mg;
wherein the methylene blue COVID-19 composition is a powder;
wherein the powder dissolves in a liquid to provide an easy method of consumption for a user; and
wherein the liquid is water.

15. The methylene blue COVID-19 composition solution, according to claim 14, wherein the powder is used as a treatment for up to about 6 months after testing positive for a COVID-19 infection.

16. The methylene blue COVID-19 composition solution, according to claim 14, wherein the quantity of methylene blue counteracts metabolic oxidative stress responsible for a pathogenesis of COVID-19.

17. The methylene blue COVID-19 composition solution, according to claim 14, wherein the methylene blue targets immune system cells and molecular mechanisms responsible for triggering an adequate body response to viral infection.

18. The methylene blue COVID-19 composition solution, according to claim 14, wherein a combination of methylene blue and aspirin have synergistic effects in treating viral infections.

19. The methylene blue COVID-19 composition solution, according to claim 14, wherein the quantity of aspirin reduces levels of a plurality of interleukin-6(IL-6) that is an immune signaling molecule associated with cytokine storm for COVID-19 patients.

20. The methylene blue COVID-19 composition, according to claim 19, wherein the methylene blue COVID-19 composition is in a pill form.
